(19) United States
(12) Patent Application Publication
(10) Pub. No.: US 2007/0088351 A1
(43) Pub. Date: Apr. 19, 2007

(54) Reusable cautery probe

(75) Inventors: Michael Ewaschuk, Haverhill, MA (US); Marc Theroux, Grafton, MA (US)

Correspondence Address: GREENBLUM & BERNSTEIN, P.L.C., 1950 ROLAND CLARKE PLACE, RESTON, VA, 20191, US

(73) Assignee: MICROLINE PENTAX Inc., Beverly, MA (US)

(21) Appl. No.: 11/249,479

(22) Filed: Oct. 14, 2005

(51) Int. Cl. A61B 18/14

(52) U.S. Cl. 606/45; 606/59

(57) ABSTRACT

A fixed tip electrode includes a hollow shaft having a proximal end and a distal end, an electrode tip assembly removably affixed to the distal end of the shaft, and a cap assembly removably affixed to the proximal end of the shaft. The hollow shaft, the electrode tip assembly and the cap assembly are all in electrically conductive communication with each other.

### BACKGROUND OF THE INVENTION

1. Field of the Invention

The present invention is related to a fixed tip electrode, and more particularly relates to a reusable fixed tip electrode for medical cauterization.

2. Background and Material Information

Cautery probes, also referred to as fixed tip electrodes, have a wide variety of surgical applications. Such probes usually include a tip affixed to a shaft, and are connectable to an electrical source such as an electrosurgical generator. After use, for hygienic purposes, such probes are generally discarded and therefore, probe tips are not interchangeable with probe shafts (as there is no need to do so, given their disposability). Additionally, different types of surgeries require different types of probes, i.e., probes having different electrical and/or mechanical properties and configurations. Thus, if a surgeon performs different types of surgeries, he or she would be required to keep a variety of different types of probes in inventory, which results in increased cost and inventory time. Therefore, a need has arisen for a reusable fixed tip electrode that can be configured to accept different types of probe tip assemblies.

### SUMMARY OF THE INVENTION

A non-limiting embodiment of the present invention provides a fixed tip electrode having a hollow shaft having a proximal end and a distal end, an electrode tip assembly removably affixed to the distal end of the shaft, and a cap assembly removably affixed to the proximal end of the shaft, wherein the hollow shaft, the electrode tip assembly and the cap assembly are all in electrically conductive communication with each other.

Another feature includes an inner luer affixed to the proximal end of the shaft, wherein the cap assembly includes an outer luer and a cautery post, the cautery post configured to connect to an electrical source, the cap assembly is removably affixed to the proximal end of the shaft via engagement of the outer luer with the inner luer.

In another feature, the outer luer and the inner luer are threadably affixed to each other, and in a further feature, the cautery post is removably affixed to the cap assembly.

According to an additional feature, the hollow shaft includes an outer tube and an electrically conductive inner shaft substantially disposed within the outer tube, and the electrode tip assembly includes an electrically conductive yoke removably attached to the inner shaft, and an electrically nonconductive back hub removably attached to the outer tube, and the electrode tip assembly is removably affixed to the distal end of the shaft via the yoke, the inner shaft, the back hub and the outer tube.

In another feature the yoke is threadably attached, via first threads, to a distal end of the inner shaft, the back hub is threadably attached, via second threads, to a distal end of the outer tube, and the first threads and the second threads have thread pitches different from each other.

Also, the cap assembly may have an adjustment knob, and the cap assembly may be threadably affixed to the proximal end of the hollow shaft via the adjustment knob, and the electrode may further have a spring configured to proximally bias the inner shaft to contact the adjustment knob.

In still another feature, rotating the adjustment knob in a first direction distally moves the inner shaft, and rotating the adjustment knob in a second direction proximally moves the inner shaft.

In yet a further feature, the distal end of the outer tube includes a semi-crystalline electrically-resistive plastic material, and the semi-crystalline electrically-resistive plastic material may include at least one polyether ether ketone (PEEK) material.

In an additional feature, the cap assembly may include a handle cap that surrounds the cautery post and proximally extends a greater amount than any proximal extension of the cautery post. Additionally, the handle cap may be made from an electrically-resistive material such as plastic, glass, rubber, latex and/or ceramic.

In yet still another feature a flushing port is located on the proximal end of the hollow shaft, the flushing port providing access to an inside of the hollow shaft, wherein the hollow shaft is exposed upon removal of the cap assembly from the shaft, and the flushing port is configured to accept attachment of a flushing nozzle thereto.

A non-limiting embodiment of the present invention provides a body for a fixed tip electrode, the body having a hollow shaft having a proximal end and a distal end, and a cap assembly removably affixed to the proximal end of the hollow shaft, wherein the distal end of the shaft is configured to removably accept the attachment of an electrode tip assembly, and the hollow shaft, the electrode tip assembly and the cap assembly are all in electrically conductive communication with each other.

In a further feature, an inner luer is affixed to the proximal end of the shaft, wherein the cap assembly includes an outer luer and a cautery post, the cautery post configured to connect to an electrical source, the cap assembly is removably affixed to the proximal end of the shaft via engagement of the outer luer with the inner luer. In another feature, the outer luer and the inner luer are threadably affixed to each other.

In an additional feature, the hollow shaft includes an outer tube configured to removably accept the attachment of an electrically nonconductive back hub of the electrode tip assembly, and an electrically conductive inner shaft substantially disposed within the outer tube and configured to removably accept the attachment of an electrically conductive yoke of the electrode tip assembly, and the distal end of the shaft is configured to removably accept the attachment of an electrode tip assembly via the attachment of the yoke to the inner shaft and the attachment of the back hub to the outer tube.

In still an another feature, a distal end of the inner shaft is configured to accept the yoke via first threads on the distal end of the inner shaft, a distal end of the outer tube is configured to accept the back hub via second threads on the distal end of the outer tube, and the first threads and the second threads have thread pitches different from each other.

In yet a further feature, the cap assembly includes an adjustment knob, and the cap assembly is threadably affixed to the proximal end of the hollow shaft via the adjustment knob, the body further having a spring configured to proximally bias the inner shaft to contact the adjustment knob. Also, rotating the adjustment knob in a first direction may distally move the inner shaft, and rotating the adjustment knob in a second direction may proximally move the inner shaft.

In an additional feature, the distal end of the shaft is configured to removably and selectively accept the attachment of any of a plurality of electrode tip assemblies each having a different mechanical configuration. Additionally, the outer luer may surround the cautery post and proximally extend a greater amount than any proximal extension of the cautery post.

In yet still another feature, a flushing port on the proximal end of the hollow shaft may be provided, the flushing port providing access to an inside of the hollow shaft, wherein the hollow shaft is exposed upon removal of the cap assembly from the shaft, and the flushing port is configured to accept attachment of a flushing nozzle thereto.

A non-limiting embodiment of the present invention further provides a method of flushing an internal lumen of a fixed tip electrode, the electrode having a hollow shaft, an electrode tip assembly removably affixed to a distal end of the shaft, and a cap assembly removably affixed to a proximal end of the shaft. The method may include removing the electrode tip assembly from the distal end of the shaft, removing the cap assembly from the proximal end of the shaft, affixing a flushing nozzle to one of the proximal end and the distal end of the shaft, and infusing, via the flushing nozzle, the hollow shaft with fluid such that the fluid exits the other of the proximal end and the distal end of the shaft.

In another feature, the affixing may include threadably affixing the flushing nozzle to the one of the proximal end and the distal end of the shaft.

Other exemplary embodiments and advantages of the present invention may be ascertained by reviewing the present disclosure and the accompanying drawings, and the above description should not be considered to limit the scope of the present invention.

### BRIEF DESCRIPTION OF THE DRAWINGS

The present invention is further described in the detailed description which follows, in reference to the noted plurality of drawings, by way of non-limiting examples of preferred embodiments of the present invention, in which like characters represent like elements throughout the several views of the drawings, and wherein:

FIG. 1 is a front perspective view of a fixed-tip electrode according to an embodiment of the present invention;

FIG. 2 is a rear perspective view of the fixed-tip electrode;

FIG. 3 is a perspective view of the electrode tip according to the embodiment of the present invention;

FIG. 4 shows a sectional view of the electrode tip disengaged from a shaft according to the embodiment of the present invention;

FIG. 5 shows a sectional view of the electrode tip engaged with a shaft according to the embodiment of the present invention;

FIG. 6 shows a sectional view of a handle assembly according to the embodiment of the present invention;

FIG. 7 shows an exploded sectional view of the handle assembly according to the embodiment of the present invention;

FIG. 8 shows an enlarged sectional view of the handle assembly according to the embodiment of the present invention; and

FIG. 9 shows a side elevational view of a cleaning device to be used with the embodiment of the present invention.

### DETAILED DESCRIPTION OF THE PREFERRED EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

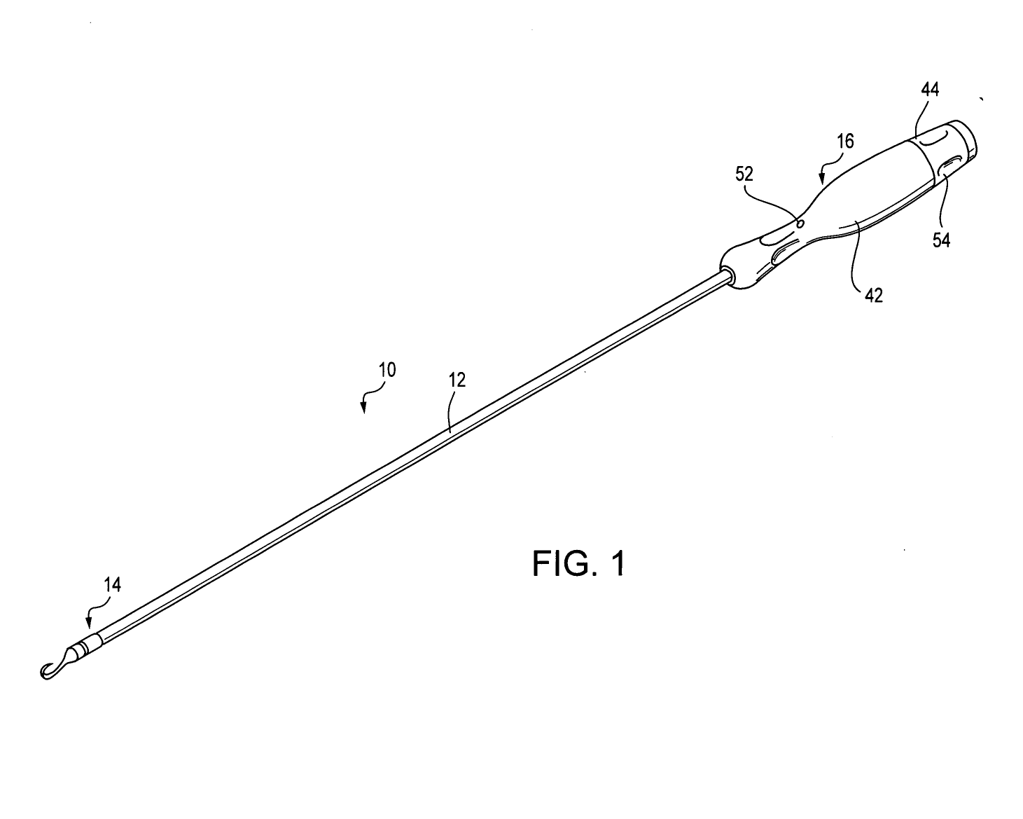

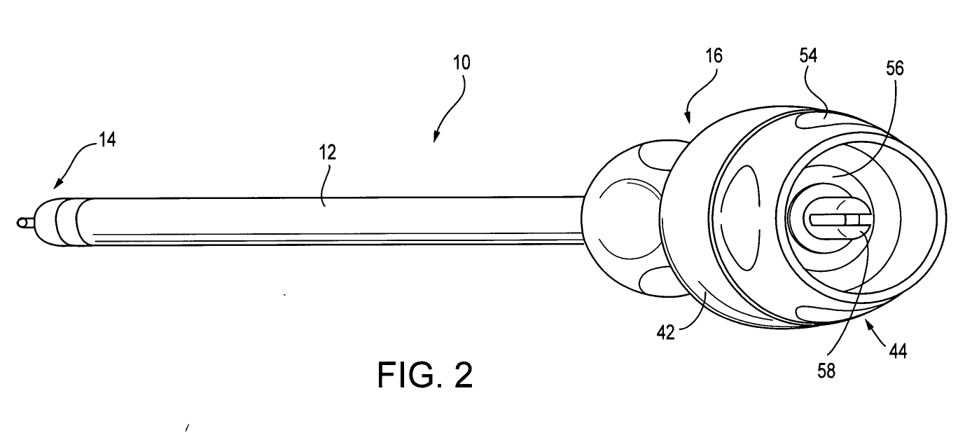

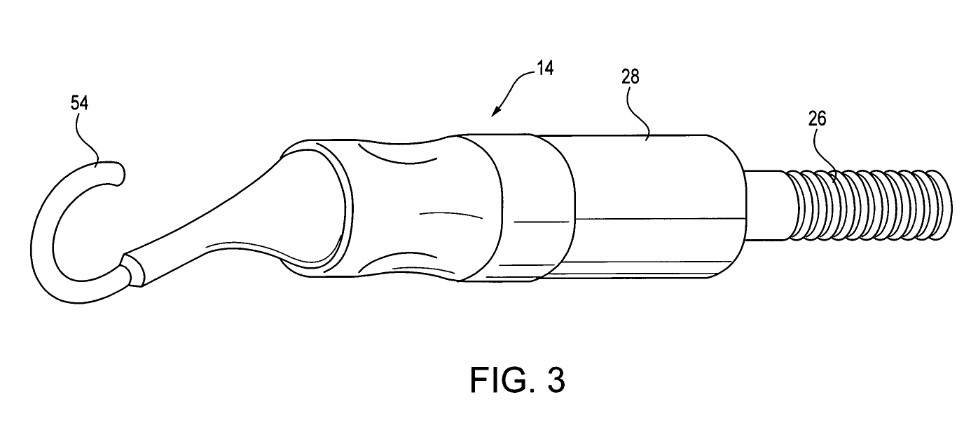

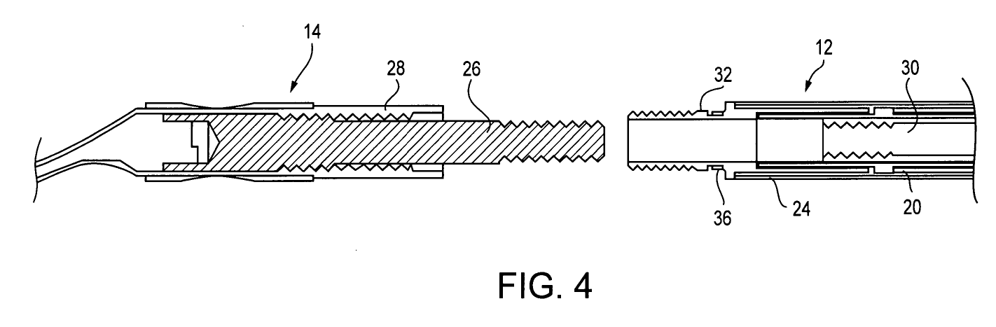

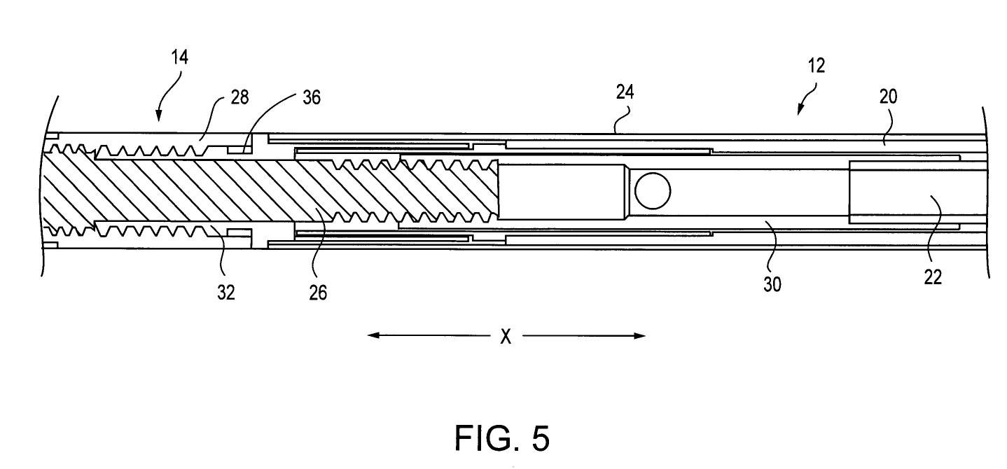

Referring to the drawings, wherein like characters represent like elements, FIGS. 1 and 2 show perspective views of a fixed-tip electrode 10 (the “device,” also called a “cautery probe”) according to an embodiment of the present invention. The electrode has a hollow shaft 12 (also referred to as a main body) having an electrode tip 14 affixed to the distal end thereof, and also has a handle assembly 16 affixed to the proximal end of the shaft 12. FIG. 3 shows the electrode tip, which is fixed, i.e., it has no moving parts. FIG. 4 shows a sectional view of the electrode tip 14 disengaged from the distal end of the shaft 12, and FIG. 5 shows a sectional view of the electrode tip engaged with the distal end of the shaft. The electrode tip 14 has a probe end 18 formed of a conductive material, and is configured to contact and cauterize tissue in a mammalian body. The probe end 18 is merely illustrative of one of many shapes and configurations that may be used with the present invention for different surgical procedures. Specifically, because the shaft 12 and handle assembly 16 are reusable, each time the shaft and handle assembly are reused, a different electrode tip 14 may be used therewith, depending on the type of surgical procedure performed.

The electrode tip 14 includes an electrically-conductive threaded yoke 26 and a threaded non-electrically-conductive back hub 28, both of which attach to the shaft 12 during the electrode tip 14 attachment process.

The shaft 12 includes an outer tube 20, an electrically-conductive inner shaft 22 coaxially disposed within the outer tube and an outer sheath 24. During the electrode tip 14 attachment process, the shaft is configured to slide within the outer tube 20 in axial direction X. Also, the inner shaft 22 at its distalmost end may include a threaded shaft adapter 30 that threadably attaches to the yoke 26 of the electrode tip 14. Alternatively, in the place of the shaft adapter 30, the inner shaft may be internally threaded to accommodate the yoke 26. The shaft 12, at its distalmost location, further includes a threaded tube end 32 that threadably attaches to the back hub 28 of the electrode tip. The tube end 32 may be formed of a semi-crystalline electrically resistive plastic material, including but not limited to polyether ether ketone (PEEK), as well as a seal 36 to prevent fluid from leaking into the device 10.

The yoke-inner shaft threading may be of a thread pitch that is different from the back hub-tube end threading (referred to as a “dual thread pitch design”), or alternatively, the thread pitches may be the same. Thus the electrode tip 14 may attach to the shaft 12 in a manner described in commonly-assigned U.S. patent application Ser. Nos. 11/188,704 and/or 11/189,789, both the contents of which are expressly incorporated by reference in their entireties.

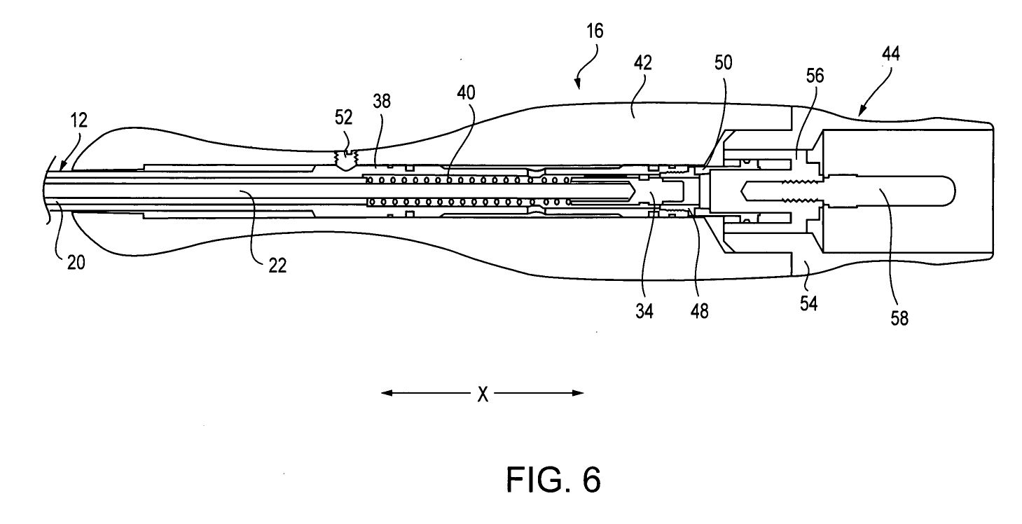

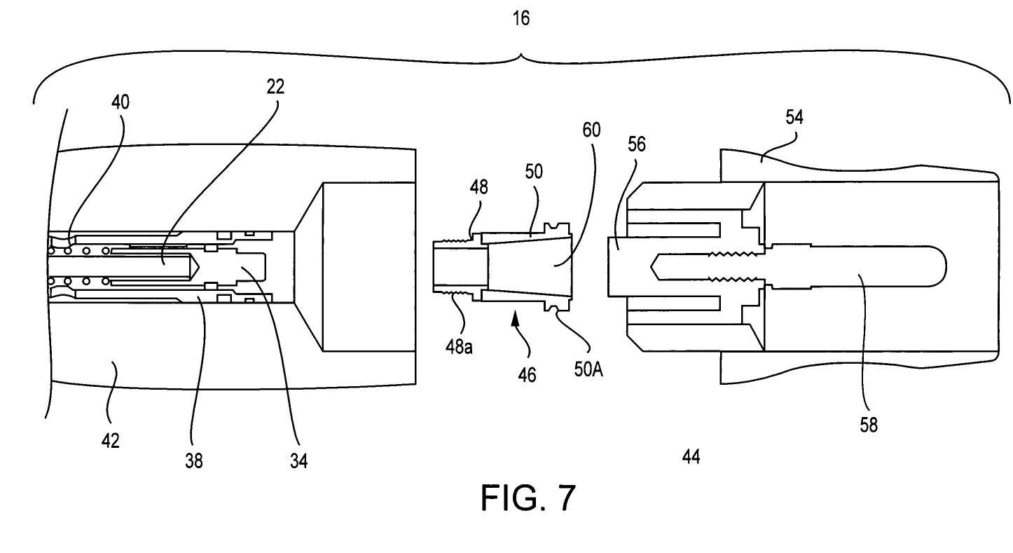

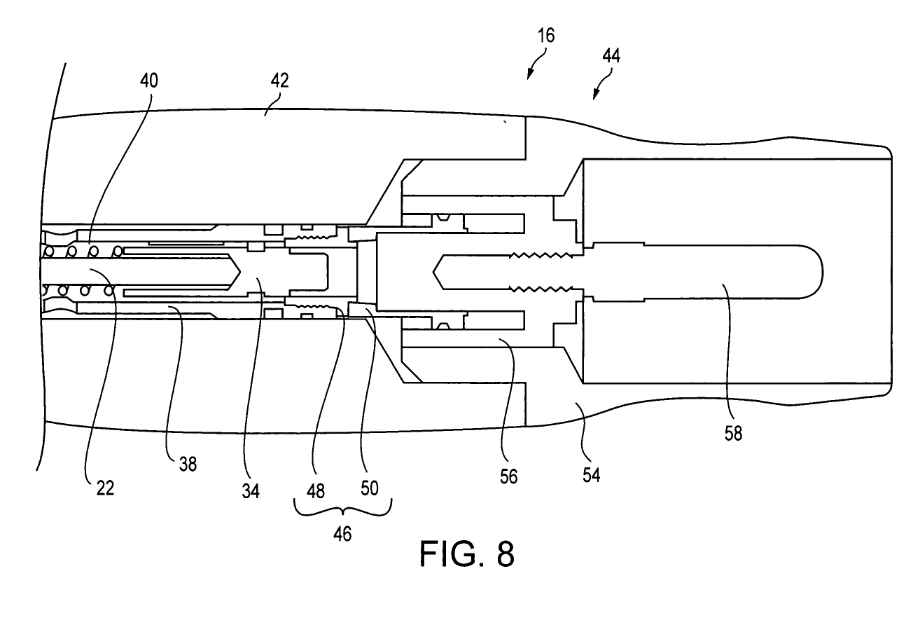

As shown in FIG. 6, at its proximalmost end, the shaft 12 includes a bearing 38 proximally extending from the outer tube 20. An electrically-conductive hollow inner luer assembly 46 is threadably affixed to the proximalmost end of the bearing 38. As shown in FIGS. 6-8, the inner luer assembly 46 includes an adjustment knob 48 and an inner luer 50. The adjustment knob 48 and inner luer 50 may be formed so that the inner luer assembly is a unitary piece (so that the inner luer assembly also functions as an adjustment knob), or alternatively, the adjustment knob and inner luer may be joined from separate components (by, e.g., laser welding or threading) to form the inner luer assembly. The inner luer assembly 46 is threaded with adjustment threads 48a that threadably engage the proximalmost end of the bearing 38 to affix the inner luer assembly 46 to the bearing. Alternatively, the inner luer assembly 46 may be integrally or unitarily formed with the bearing 38. The inner luer assembly 46 also includes inner luer threads 50A to accept the threaded attachment of a cap assembly 44 to create a fluid-resistive seal due to the configuration of their respective mating surfaces. Additionally or alternatively, an O-ring, grease and/or compliant sealing material may be used at the mating surfaces of the cap assembly 44 and inner luer assembly to increase the fluid-resistive characteristics thereof.

A coil spring 40 is disposed about the inner shaft 22 between the proximalmost end of the outer tube 20 and a shaft bushing 34 located on the proximalmost end of the inner shaft. With this arrangement, the coil spring 40 proximally biases the inner shaft 22 along the axial direction X such that the shaft bushing 34 engages the adjustment knob 48.

In situations where the yoke-inner shaft threading is of a thread pitch that is different from the back hub-tube end threading (i.e., in a “dual thread pitch design”), the inner shaft 22 travels in the axial direction X. Further, since the shaft of the present invention may be used with a variety of electrode tips 14 having different structural configurations (including different thread pitch configurations), the distance traveled by the inner shaft 22 along axial direction X will differ with each type of electrode tip 14 affixed to the shaft 12. Due to the presence of the coil spring 40 which removes play and biases the shaft bushing 34 toward the adjustment knob 48, the position in the axial direction X of the inner shaft 22 may be adjusted and fixed by rotating the inner luer assembly 46 and/or adjustment knob 48, thereby securing the electrode tip 14 in place, irrespective of the structural configuration of the various electrode tips 14 to be connected to the shaft 12.

It is also noted that due to the dual thread pitch design of the present invention, the present invention may be used in conjunction with the movable tools (e.g., graspers, shears, etc.) described in commonly-assigned U.S. patent application Ser. Nos. 11/188,704 and/or 11/189,789; however, the movable handles in these tools will not move when used in conjunction with the present invention, since the present invention uses a fixed tip electrode, which does not move during surgery.

As shown in FIGS. 6-9, the handle assembly 16 includes a handle 42 and a cap assembly 44. As shown in FIG. 6, the handle 42 is affixed about the proximal end of the shaft 12, and, using a set screw 52 that engages a detent in the bearing 38, may be prevented from sliding in the axial direction X and from rotating about the shaft. The cap assembly 44 includes a generally tubular non-electrically-conductive handle cap 54 that surrounds an electrically-conductive outer luer 56 and an electrically-conductive cautery post 58. The cap 54 may be made of any suitable electrically-resistive material, including but not limited to plastic, rubber, latex, glass and ceramic. Although the figures show the cautery post 58 threadably inserted into the outer luer 56 (i.e., they are shown as two separate components), in alternative embodiments the cautery post and outer luer may be unitarily formed. The cautery post 58 is configured to be plugged into a cord of an electrical source (e.g., an electrosurgical generator), and is protected by the cap 54 that surrounds the cautery post. As shown in FIGS. 6-8, to further protect the cautery post 58 from being damaged due to an external force, as well as to provide for added safety to the surgeon, the cap 54 protrudes in a proximal direction by an amount greater than the proximal protrusion of the cautery post.

The cap assembly 44 is affixed to the proximal end of the shaft 12 by connecting to the inner luer assembly 46 via threads (not shown) on an inner surface of the outer luer 56, which engage the inner luer threads 50A. Additionally, at least a portion of the cap 54 is housed within the handle 42 to create a fluid-resistive seal and to further secure the cap assembly 44 against movement in the lateral direction, which could otherwise damage the device. It should be appreciated by those skilled in the art, as further described hereinbelow, that the cap assembly need not threadably be attached to the shaft, but may be attached via alternative methods, including but not limited to press-fitting, bayonet-type attachments, spring-loaded ball detents, and locking rings. Additionally, the cap assembly may not include an outer luer; rather, the cautery post 58 and/or the cap 54 may attach directly to the inner luer assembly 46 via any of the aforementioned methods.

Once the cautery post 58 of the device 10 is plugged into an active electrical source (not shown), electrical current flows to the probe end 18 via the cautery post 58, outer luer 54, inner luer assembly 46, inner shaft 22 and yoke 26. These elements, as well as the other conductive elements of the device 10 may be made of stainless steel or any other suitable conductive material. Additionally, the conductive elements may be coated on certain areas to facilitate the control and application of electrical current during surgery.

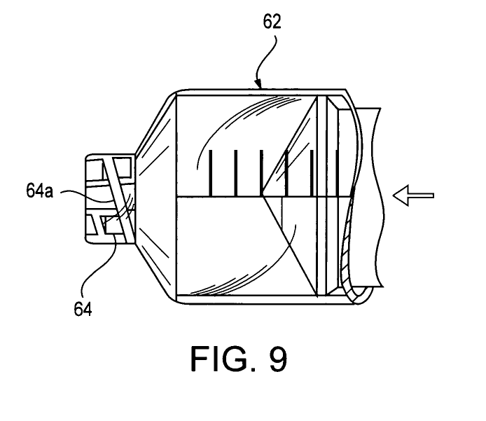

After surgery, the device 10 may be reused by flushing the lumen (i.e., the inner surfaces) thereof. The sterilization process may be performed as follows: Initially, in no particular order, the electrode tip 14 and cap assembly 44 are removed from the shaft 12. Specifically, the electrode tip 14 is removed from the shaft 12 by decoupling the yoke 26 from the inner shaft 22 and by decoupling the back hub 28 from the tube end 32. The electrode tip may then be sterilized (e.g., by autoclave) or discarded. The cap assembly 44 is removed from the shaft 12 by decoupling the outer luer 56 from the inner luer threads 50A to expose the lumen to ambient via the hollow inner luer assembly 46. In this regard, the proximal end of the inner luer 50 functions as a flushing port 60. Conversely, when the cap assembly is affixed to the shaft 12, the outer luer 56 covers the flushing port 60 to seal the proximal end of the lumen. Once the cap assembly 44 has been removed, the cap assembly may be sterilized (e.g., by autoclave) or discarded, and a flushing device 62 (shown in FIG. 9), including but not limited to a syringe having a flushing nozzle 64, is affixed to the inner luer assembly 46 by threadably engaging nozzle threads 64a with the inner luer threads 50A. In this manner, a fluid-resistant seal is formed between the flushing device 62 and the flushing port 60, whereupon cleaning fluid may be introduced into the lumen to clean the shaft 12. It is also noted that the distal end of the shaft 12 may alternatively or additionally function as a flushing port by allowing the flushing nozzle 64 to attach to the tube end 32 and or the inner shaft 22. Once cleaned, the device 10 may be reused by reattaching the cap assembly to the shaft 12 and by reattaching or attaching a new electrode tip 14 to the shaft.

It is noted that while threads are used to describe the attachment of certain elements of the present invention (e.g., yoke-inner shaft, back hub-tube end, outer luer-inner luer, inner luer-bearing), it should be readily understood by those skilled in the art that alternative types of attachment may be used, including but not limited to, for example, press-fittings, bayonet-type attachments, spring-loaded ball detents, locking rings, and other attachment types described in commonly-assigned U.S. patent application Ser. No. 11/179,509, the contents of which are expressly incorporated by reference in its entirety.

It is further noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A fixed tip electrode comprising:
a hollow shaft having a proximal end and a distal end;
an electrode tip assembly removably affixed to said distal end of said shaft; and
a cap assembly removably affixed to said proximal end of said shaft;
wherein said hollow shaft, said electrode tip assembly and said cap assembly are all in electrically conductive communication with each other.

2. The electrode according to claim 1, further comprising an inner luer affixed to said proximal end of said shaft,
wherein:
said cap assembly comprises an outer luer and a cautery post, said cautery post configured to connect to an electrical source;
said cap assembly is removably affixed to said proximal end of said shaft via engagement of said outer luer with said inner luer.

3. The electrode according to claim 2, wherein said outer luer and said inner luer are threadably affixed to each other.

4. The electrode according to claim 2, wherein said cautery post is removably affixed to said cap assembly.

5. The electrode according to claim 1, wherein:
said hollow shaft comprises an outer tube and an electrically conductive inner shaft substantially disposed within said outer tube;
said electrode tip assembly comprises:
an electrically conductive yoke removably attached to said inner shaft; and
an electrically nonconductive back hub removably attached to said outer tube; and
said electrode tip assembly is removably affixed to said distal end of said shaft via said yoke, said inner shaft, said back hub and said outer tube.

6. The electrode according to claim 5, wherein:
said yoke is threadably attached, via first threads, to a distal end of said inner shaft;
said back hub is threadably attached, via second threads, to a distal end of said outer tube; and
said first threads and said second threads have thread pitches different from each other.

7. The electrode according to claim 5, wherein:
said cap assembly comprises an adjustment knob; and
said cap assembly is threadably affixed to said proximal end of said hollow shaft via said adjustment knob,
the electrode further comprising a spring configured to proximally bias said inner shaft to contact said adjustment knob.

8. The electrode according to claim 7, wherein:
rotating said adjustment knob in a first direction distally moves said inner shaft; and
rotating said adjustment knob in a second direction proximally moves said inner shaft.

9. The electrode according to claim 5, wherein said distal end of said outer tube comprises a semi-crystalline electrically-resistive plastic material.

10. The electrode according to claim 9, wherein said semi-crystalline electrically-resistive plastic material includes at least one polyether ether ketone material.

11. The electrode according to claim 2, wherein said outer luer surrounds said cautery post and proximally extends a greater amount than any proximal extension of said cautery post.

12. The electrode according to claim 1, further comprising a flushing port on the proximal end of said hollow shaft, said flushing port providing access to an inside of said hollow shaft, wherein:
said hollow shaft is exposed upon removal of said cap assembly from said shaft; and
said flushing port is configured to accept attachment of a flushing nozzle thereto.

13. A body for a fixed tip electrode, the body comprising:
a hollow shaft having a proximal end and a distal end; and
a cap assembly removably affixed to said proximal end of said hollow shaft;
wherein:
said distal end of said shaft is configured to removably accept the attachment of an electrode tip assembly; and
said hollow shaft, the electrode tip assembly and said cap assembly are all in electrically conductive communication with each other.

14. The body according to claim 13, further comprising
an inner luer affixed to said proximal end of said shaft, wherein:
said cap assembly comprises an outer luer and a cautery post, said cautery post configured to connect to an electrical source;
said cap assembly is removably affixed to said proximal end of said shaft via engagement of said outer luer with said inner luer.

15. The body according to claim 14, wherein said outer luer and said inner luer are threadably affixed to each other.

16. The body according to claim 14, wherein said cautery post is removably affixed to said cap assembly.

17. The body according to claim 13, wherein:
said hollow shaft comprises:
an outer tube configured to removably accept the attachment of an electrically nonconductive back hub of the electrode tip assembly; and
an electrically conductive inner shaft substantially disposed within said outer tube and configured to removably accept the attachment of an electrically conductive yoke of the electrode tip assembly; and
said distal end of said shaft is configured to removably accept the attachment of an electrode tip assembly via the attachment of the yoke to the inner shaft and the attachment of the back hub to the outer tube.

18. The body according to claim 17, wherein:
a distal end of said inner shaft is configured to accept the yoke via first threads on said distal end of said inner shaft;
a distal end of said outer tube is configured to accept the back hub via second threads on the distal end of said outer tube; and
said first threads and said second threads have thread pitches different from each other.

19. The body according to claim 17, wherein:
said cap assembly comprises an adjustment knob; and
said cap assembly is threadably affixed to said proximal end of said hollow shaft via said adjustment knob,
the body further comprising a spring configured to proximally bias said inner shaft to contact said adjustment knob.

20. The body according to claim 19, wherein:
rotating said adjustment knob in a first direction distally moves said inner shaft; and
rotating said adjustment knob in a second direction proximally moves said inner shaft.

21. The body according to claim 17, wherein said distal end of said shaft is configured to removably and selectively accept the attachment of any of a plurality of electrode tip assemblies each having a different mechanical configuration.

22. The body according to claim 14, wherein said cap assembly further comprises a handle cap surrounding said cautery post and proximally extending a greater amount than any proximal extension of said cautery post.

23. The body according to claim 13, further comprising a flushing port on the proximal end of said hollow shaft, said flushing port providing access to an inside of said hollow shaft, wherein:
said hollow shaft is exposed upon removal of said cap assembly from said shaft; and
said flushing port is configured to accept attachment of a flushing nozzle thereto.

24. The body according to claim 22, wherein said handle cap comprises an electrically resistive material.

25. The body according to claim 24, wherein said electrically resistive material is one of plastic, glass, rubber, latex or ceramic.

26. A method of flushing an internal lumen of a fixed tip electrode, the electrode having a hollow shaft, an electrode tip assembly removably affixed to a distal end of the shaft; and a cap assembly removably affixed to a proximal end of the shaft, the method comprising:
removing the electrode tip assembly from the distal end of the shaft;
removing the cap assembly from the proximal end of the shaft;
affixing a flushing nozzle to one of the proximal end and the distal end of the shaft; and
infusing, via the flushing nozzle, the hollow shaft with fluid such that the fluid exits the other of the proximal end and the distal end of the shaft.

27. The method according to claim 26, wherein said affixing comprises threadably affixing the flushing nozzle to the one of the proximal end and the distal end of the shaft.

28. The method according to claim 26, further comprising expelling the fluid from the other of the proximal end and the distal end of the shaft
